(19) United States
(12) Patent Application Publication
(10) Pub. No.: US 2020/0121253 A1
(43) Pub. Date: Apr. 23, 2020

(54) Medical Examination Table for Babies

(71) Applicant: Fama Sofas, S.L., Yecla (ES)

(72) Inventors: Paloma López Martínez, Yecla (ES); Félix López Gil, Yecla (ES)

(21) Appl. No.: 16/493,980

(22) PCT Filed: Feb. 12, 2018
(86) PCT No.: PCT/ES2018/070097

§ 371 Date: Sep. 13, 2019

(30) Foreign Application Priority Data
Mar. 15, 2017 (ES) U201730269

(51) Int. Cl. A61B 5/00

(52) U.S. Cl. CPC A61B 5/704; A61B 2503/04

(57) ABSTRACT

The invention relates to a medical examination table (1) for babies, comprising a body (2) having an upper surface (3) adapted anatomically to receive a baby lying down, and a lower face (4) comprising a projection (5) adapted to the legs (100) of the doctor.

### OBJECT OF THE INVENTION

The present invention relates to a table for any type of minor outpatient and hospital medical examinations for babies, such as examinations of the eyes, nose, or mouth, as well as physical therapy and rehabilitation treatments for babies in the body and limbs.

### BACKGROUND OF THE INVENTION

Today there are minor outpatient-type medical examinations for babies that can be performed, for example, in the fields of otorhinolaryngology or dentistry, that do not offer suitable support for the babies, because the medical examination seats or chairs in these specialty fields and other similar ones are conceived for adults and even children, such that they can be adjusted in height, but not for babies who cannot sit upright or who may make involuntary movements during the medical examination.

These drawbacks are solved with the use of the table of the invention.

### DESCRIPTION OF THE INVENTION

The table of the invention has a configuration which allows placing the baby comfortably on the legs of the doctor during a medical examination, therefore in an optimal and up-close position, and with a simple and economical configuration.

According to the invention, the table comprises a body having an upper surface adapted anatomically to receive a baby lying down, and a lower face comprising a projection adapted to the legs of the doctor. This projection thereby fits on the legs of the doctor, ensuring perfect seating of the table, while the upper part is used for receiving the baby lying down.

### DESCRIPTION OF THE DRAWINGS

FIGS. 1 to 3 show respective views of the table of the invention from different angles.

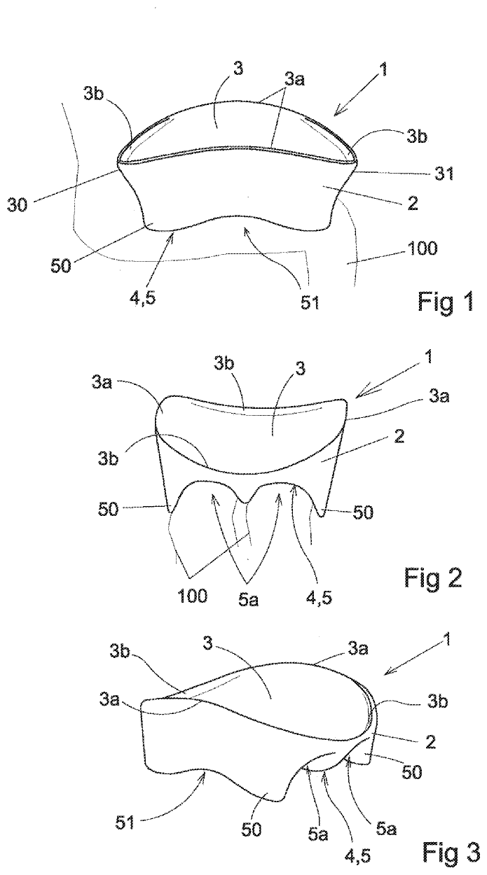

### PREFERRED EMBODIMENT OF THE INVENTION

The medical examination table (1) for babies of the invention comprises a body (2) having an upper surface (3) adapted anatomically to receive a baby lying down, and a lower face (4) comprising a projection (5) adapted to the legs (100) of the doctor.

The upper surface (3) comprises an upwardly oriented concave cross-section, which allows the baby to be protected and held, preventing voluntary or involuntary lateral movements and slipping out. Furthermore, the upper surface (3) comprises central areas (3a) with a greater height on both sides, because greater protection is required in this area as it comprises the baby's torso, which has a higher inertia for slipping out because it weighs more, leaving more space at the end zones corresponding to the head or lower limbs so that the doctor can work comfortably. It has also been envisaged that the upper surface (3) preferably comprises ascending edges (3b) at the proximal end (30) and distal end (31) thereof for holding a cushion, not depicted.

In turn, the projection (5) adapted to the legs of the doctor of the lower face (5) of the body (2) ideally comprises a cross-section with two downwardly oriented adjacent depressions (5a) in which both legs (100) of the doctor fit. Preferably, the outer sides (50) of the two depressions (5a) comprise in the central part thereof an area (51) having a smaller height than at the end parts thereof to increase the doctor's comfort.

In the preferred configuration, the body (2) has a single-piece configuration and allows being manufactured from plastics.

Having sufficiently described the nature of the invention as well as the manner of carrying it out to practice, it must be stated that the arrangements indicated above and depicted in the attached drawings are susceptible to modifications in detail provided that they do not alter the fundamental principle thereof.

1. A medical examination table for babies, comprising a body having an upper surface k adapted anatomically to receive a baby lying down, and a lower face comprising a projection adapted to the legs of the doctor.

2. The medical examination table for babies according to claim 1, wherein the upper surface comprises an upwardly oriented concave cross-section.

3. The medical examination table for babies according to claim 2, wherein the upper surface comprises central areas with a greater height on both sides.

4. The medical examination table for babies according to claim 1, wherein the upper surface comprises ascending edges at a proximal end and distal end thereof for holding a cushion.

5. The medical examination table for babies according to claim 1, wherein the projection adapted to the legs of the doctor of the lower face of the body comprises a cross-section with two downwardly oriented adjacent depressions.

6. The medical examination table for babies according to claim 5, wherein the outer sides the two depressions comprise in the central part thereof an area having a smaller height than at the end parts thereof.

7. The medical examination table for babies according to claim 1, wherein the body has a single-piece configuration.

8. The medical examination table for babies according to claim 1, wherein the body is made from plastics.
